SEQUENCE LISTING (2 sequences)

SEQ ID NO: 1 - protein, 9 aa, Artificial Sequence
  Description of Artificial Sequence Synthetic proteolytic cleavage peptide
RKRRKRRKR

SEQ ID NO: 2 - protein, 8 aa, Artificial Sequence
  Description of Artificial Sequence Synthetic proteolytic cleavage peptide
PRPSRKRR

(19) United States
(12) Patent Application Publication
(10) Pub. No.: US 2004/0102388 A1
(43) Pub. Date: May 27, 2004

(54) Modified blood clotting factors and methods of use

(76) Inventors: Katherine A. High, Merion, PA (US); Paris Margaritis, Philadelphia, PA (US); Rodney Camire, Voorhees, NJ (US)

Correspondence Address: Robert Bedgood, Pillsbury Winthrop LLP, 101 W Broadway, Suite 1800, San Diego, CA, 92101, US

(21) Appl. No.: 09/816,688

(22) Filed: Mar. 22, 2001

Related U.S. Application Data
(60) Provisional application No. 60/191,331, filed on Mar. 22, 2000.

(51) Int. Cl. A61K 48/00

(52) U.S. Cl. 514/44

(57) ABSTRACT

The invention provides compositions including modified blood clotting factors that have a non-native proteolytic cleavage site engineered into them allowing intracellular cleavage and secretion of an active form. The compositions are useful in the methods for treating a bleeding or clotting disorder.

### PRIORITY CLAIM

This application claims priority to U.S. Application Serial No. 60/191,331, filed Mar. 22, 2000.

### STATEMENT AS TO FEDERALLY SPONSORED RESEARCH

The invention was made with Government support from the National Heart, Lung and Blood Institute of the National Institute of Health, Grant Nos. U01HL66948 and P01HL64190. The government may have certain rights in the invention.

### TECHNICAL FIELD

The invention relates to modified proteins having non-native proteolytic cleavage/recognition sites, and more particularly to modified blood clotting factors and methods of treating bleeding or clotting disorders.

### BACKGROUND

Blood coagulation is a mechanism by which sequential activation of zymogens by limited proteolysis leads to thrombin generation which in turn converts soluble fibrinogen to a fibrin clot (Roberts and Tabares (ed.) In: Molecular Basis of Thrombosis and Hemostasis, Marcell Dekker, Inc. NY 35-50 (1995)). The blood coagulation cascade is initiated by vascular damage, which produced tissue factor (TF). When Factor VII is complexed with TF, it is rapidly converted to FVIIa (a two-chain molecule) by cleavage at the Arg 152-Ile 153 site, generating an N-terminal light and a C-terminal heavy chain linked by a disulfide bond. FVIIa is normally present at a concentration of approximately 1% of FVII (Wildgoose, et al., Blood 80:25 (1992)). The TF-FVIIa complex activates Factor IX to Factor IXa and Factor X to Factor Xa. Factor Xa generation leads to cleavage of prothrombin to thrombin, which then cleaves soluble fibrinogen to fibrin. Thrombin maintains the generation of FXa by positive feedback. Together with fibrin, platelet aggregation results in an insoluble clot.

Mature FVII is a single-chain, 406 amino acid, vitamin-K dependent serine protease. The primary structure of FVII consists of a signal peptide, a propeptide, a Gla domain, two EGF domains and a catalytic domain. Factor VII circulates in plasma as a single chain zymogen (Mᵣ=50,000) at a concentration of 500 ng/mL (10 nM) and becomes active following cleavage of a single peptide bond (Arg¹⁵²-Ile¹⁵³). The signal peptide and propeptide are removed during transport through the cellular secretory pathway and FVII undergoes several additional post-translational modifications: (1) it is γ-carboxylated at 10 Gla residues in the Gla domain; (2) it is N-glycosylated at two asparagine sites at positions 145 and 322 (Thim, et al., Biochemistry 27:7785 (1988)); and (3) it is O-glycosylated at Ser 52 and Ser 60 (Bjoern, et al., J. Biol. Chem. 266:11051 (1991)). The resulting Factor VIIa is composed of a light chain (Mᵣ=20,000) and a heavy chain (Mᵣ=30,000) associated through a disulfide bond.

Hemophilia is an X-linked recessive disorder with a clinical phenotype of recurrent bleeding episodes, mostly into joints and soft tissues. These bleeding episodes can be life threatening, especially when they occur in closed spaces such as the intracranial space or the retroperitoneum. Genetically, there are two types of hemophilia, A and B, clinically indistinguishable. Hemophilia A is caused by mutations in the gene for Factor VIII. Mutations in the gene for Factor IX are responsible for hemophilia B. Affected individuals exhibit varying levels of disease severity, which correlate closely with their circulating levels of Factor VIII or Factor IX (normal levels for Factor VIII are 100-200 ng/ml; for Factor IX: ˜5 μg/ml). Hemophilia patients with <1% circulating levels are classified as severe, those with 1-5% are moderate and those with >5% circulating levels are mildly affected. The prevalence of hemophilia A is approximately 4-5 fold higher than that of hemophilia B.

Hemophiliacs have been traditionally treated with intravenous infusions of clotting factor concentrates purified from human plasma. During the 1970s and 1980s, due to the lack of advanced viral screening and inactivation methods, many patients were infected with blood-borne viral agents, such as hepatitis B and C and HIV. These unfortunate events gave great impetus to the development of recombinant methods for the production of clotting factor concentrates, now commonly used for hemophilia A and B treatment. Of course, such recombinant products are expensive.

A major obstacle to treatment with infused clotting factor is the development of long-term inhibitory antibodies (inhibitors) against the clotting factor. Such patients represent about 15% and 1-3% of the hemophilia A and B population respectively, and are commonly classified as low or high titer inhibitor patients. Inhibitory antibody formation can be described as the recognition of the infused factor as a foreign antigen, but the underlying mechanism of this process is not well understood. Possible factors include the underlying mutation such as missense mutations versus large deletions in FIX, (Green, et al., Blood Coag. Fibrinol. 2:539 (1991), Green, et al., Brit. J. Haematol. 78:390 (1991)), the manufacture of the clotting factor product itself (Vermylen and Peerlinck, European Journal of Haematology Supplement 63:15-17 (1998)) and even genetic factors influencing a patients immune response (Hoyer, Brit. J Haematol. 90:498 (1995), Gill, Thromb. and Haemost. 82:500 (1999)). Of course, such products are expensive.

Treatment of patients having inhibitory antibodies for bleeding episodes require the neutralization of the inhibitor prior to administering a hemostatically effective dose of FVIII or FIX. The neutralization dose depends on the inhibitor titer, hematocrit and body weight (Hedner, Thromb. Haemost. 82:531 (1999)). The hemostatically effective FVIII or FIX after-dose may be given in conjunction with immunosuppression, if the patient has previously exhibited increased inhibitor titer after administration of FVIII or FIX (Hedner, Thromb. Haemost. 82:531 (1999)). Alternative procedures to induce hemostasis independent of FVIII or FIX include the use of prothrombin complex concentrates (PCCs), activated PCCs (aPCCs) or, in the case of hemophilia A, porcine FVIII, which has been shown to improve hemostasis but only in 50-60% of inhibitor cases (Lusher, et al., New Engl. J. Med. 303:421 (1980)).

Additionally, a number of procedures have been proposed in order to induce immunologic tolerance to factor VIII or factor IX and thus eradicate the inhibitors to these factors. For example, a repeated combination of high dose of clotting factor and immunosuppressive treatment (cyclophosphamide) in inhibitor patients has been shown to convert high responders to low responders or to induce partial immunologic tolerance (Hedner and Nilsson, Acta Med Scand 214:191 (1983)), Hedner and Tengborn, Thromb. Haemost. 54:776 (1985)).

The administration of high levels of recombinant FVIIa (rFVIIa) has been shown to have a hemostatic effect in both hemophilia A and B patients, as well as patients with inhibitors (Hedner, Thromb. Haemost. 82:531 (1999)). The hemostatically effective dose is within the range of 70-120 μg/Kg, (to achieve circulating levels of 2-4 μg/ml, Hedner, Thromb. Haemost. 82:531 1999), and is administered every 2 hours. Recombinant FVIIa (NovoSeven FVIIa) has been used in over 2,400 bleeding episodes, mostly involving inhibitor patients (Hedner, Thromb. Haemost. 82:531 (1999)). Treatment with fixed-dose injections of 90 μg/Kg was more than 90% effective for joint and muscle bleeds (Key, et al., Thromb. Haemost. 80:912 (1998)). rFVIa has also been used in major surgery of hemophilia patients with inhibitors and has shown 81% effectiveness (Lusher, et al., Blood Coag. Fibrinol. 9:119 (1998)), whereas 86% effectiveness was shown for minor surgery of such patients (Lusher, et al., Blood Coag. Fibrinol. 9:119 (1998)). Recombinant FVIIa (rFVIIa) has also been used in patients with acquired hemophilia with similar effectiveness (Hay, et al., Thromb. Haemost. 78:1463 (1997)). Experience with continuous infusion of rFVIIa has been limited but it has been shown to be as effective as repeated injection treatment, while reducing the overall dose required to maintain hemostasis (Vermylen and Peerlinck, European Journal of Haematology Supplement 63:15 (1998)), Schulman, et al, Blood Coag. Fibrinol. 9Suppl 1:S97 (1998)). However, there are many disadvantages to rFVIIa treatment including the short half life of the protein requiring repeated or continuous administration and the very high cost.

### SUMMARY OF THE INVENTION

The invention is based, at least in part, on engineering proteins in order to provide them with advantageous properties. Introduction of a proteolytic cleavage site into a blood clotting factor allows secretion of the active form from a cell that expresses the modified factor. Secretion of the cleaved factor obviates a need for proteolytic cleavage during the blood clotting process.

In one embodiment, a composition comprising a recombinant nucleic acid that encodes a modified blood clotting factor, wherein the modification comprises a proteolytic cleavage site not normally present in the factor, and wherein the factor is cleaved at the cleavage site when expressed in an animal cell, is provided. In another embodiment, a composition comprising a modified blood clotting factor polypeptide, wherein the modification comprises a proteolytic cleavage site not normally present in the factor, and wherein the factor is cleaved at the cleavage site when expressed in an animal cell, is also provided.

Compositions comprising nucleic acids and polypeptides include modified blood clotting factors of mammalian origin, e.g., primate, human, canine, feline, porcine, equine or bovine.

Modified blood clotting factors include, for example, a functional variant or a functional subsequence of a naturally occurring blood clotting factor. Modified blood clotting factors also include, for example, vitamin K-dependent procoagulents, such as Factor VII, Factor IX or Factor X and vitamin K-dependent anticoagulents such as protein C.

In additional embodiments, a modified blood clotting factor has a mammalian amino acid sequence proteolytic cleavage site. Exemplary proteolytic cleavage sites include, for example, a PACE/furin amino acid sequence, or functional variant thereof.

In further embodiments, a modified blood clotting factor has a proteolytic cleavage site including a plurality of basic amino acid sequences or a viral amino acid sequence cleavage site, such as a retroviral protein (e.g., envelope protein). In particular embodiments, a proteolytic cleavage site comprises an Arg-Lys-Arg, Arg-Lys-Arg-Arg-Lys-Arg (SEQ ID NO:1) or an PRPSRKRR (SEQ ID NO:2) sequence.

In other embodiments, a modified blood clotting factor has a proteolytic cleavage site introduced in place of its native proteolytic cleavage site. In particular aspects, modified Factor VII has a proteolytic cleavage site introduced between amino acids 152 and 153, between arginine 152 and isoleucine 153 of Factor VII, or at a position such that cleavage at the site produces a Factor VIIa having an amino-terminal isoleucine.

Functional variants and subsequences of modified blood clotting factors are also provided. In one embodiment, a variant modified blood clotting factor has one or more conservative amino acid substitutions of wild type blood clotting factor.

Variant modified blood clotting factors having altered or enhanced functions or activities as compared to a wild type blood clotting factor are also provided. In one embodiment, a variant modified blood clotting factor comprises a Factor VII having increased activity relative to wild type Factor VII. In another embodiment, a variant modified blood clotting factor comprises a Factor VII having increased stability in vivo relative to wild type Factor VII. In yet another embodiment, a variant modified blood clotting factor comprises a Factor VII having decreased immunogenicity relative to wild type Factor VII.

Cells including the modified blood clotting factors and nucleic acids encoding the modified blood clotting factors are also provided. In one embodiment, the cell is an animal cell. In various aspects, the animal cell is a mammalian cell, e.g., a human cell. In additional aspects, the animal cell is a muscle, liver, kidney or blood vessel cell. In another embodiment, the cell is present in a subject. In one aspect, the subject is a non-human transgenic animal. In another aspect, the subject is a human.

Recombinant polynucleotides further include nucleic acids encoding a modified blood clotting factor operatively linked to a regulatable or tissue specific expression control element. In one embodiment, the regulatable or tissue specific expression control element comprises a promoter. In a particular aspect, the promoter comprises a skeletal muscle actin promoter or a muscle creatine kinase promoter. In another aspect, the tissue-specific expression control element confers expression of the modified blood clotting factor in muscle, liver, kidney or blood vessel endothelium. In yet another aspect, the regulatable expression control element comprises elongation factor 1α promoter.

Recombinant polynucleotides further include vectors (e.g., cloning or expression). In one embodiment, the vector comprises a vector suitable for introduction into a cell in vivo. In another embodiment, the vector comprises an adeno-associated virus (AAV), adenovirus, retrovirus, parvovirus, papilloma virus, reovirus, rotavirus or a herpes virus. In still another embodiment, the vector comprises a plasmid vector.

Kits comprising modified blood clotting factors and nucleic acids encoding modified blood clotting factors are also provided. In one embodiment, a kit includes a modified blood clotting factor or a nucleic acid encoding a modified blood clotting factor and instructions for using or expressing the modified blood clotting factor in vitro, ex vivo or in vivo.

Pharmaceutical compositions comprising a modified blood clotting factors or a nucleic acid encoding a modified blood clotting factor and a pharmaceutically acceptable carrier are also provided.

Methods for treating a bleeding or clotting disorder of a subject having or at risk of having a bleeding or clotting disorder are also provided. In one embodiment, a method includes administering to a subject an amount of a modified blood clotting factor sufficient to ameliorate one or more symptoms of the disorder. In another embodiment, a method includes administering to a subject an amount of a nucleic acid encoding a modified blood clotting factor sufficient to ameliorate one or more symptoms of the disorder. In yet another embodiment, a bleeding or clotting disorder is amenable to treatment with Factor VII, Factor VIII or Factor IX. In still another embodiment, a bleeding or clotting disorder is caused by insufficient activity or expression of a vitamin-K dependent procoagulent. In an additional embodiment, a bleeding or clotting disorder is caused by insufficient platelet aggregation.

In still further embodiments, the disorder comprises hemophilia, e.g., hemophilia A or hemophilia B; or a Factor VII deficiency. In still additional embodiments, the disorder comprises Glanzmann's thrombasthenia or Bemard-Soulier's thrombasthenia.

The methods of the invention include embodiments for treating a subject that produces inhibitors of a clotting factor, such as inhibitory antibodies that bind to a clotting factor. In one embodiment, a subject produces inhibitory antibodies that bind Factor VIII or Factor IX.

Methods for treating a bleeding or clotting disorder of a subject having or at risk of having a bleeding or clotting disorder, wherein the subject is a mammal such as a human are also provided.

Methods for treating a subject include administering a composition by injection or infusion. In other embodiments, a composition is administered into the portal vein or spleen.

Methods of decreasing clotting time in a subject in need of decreased clotting time are also provided. In one embodiment, a method includes administering to a subject an amount of a modified blood clotting factor sufficient to decrease clotting time in the subject. In another embodiment, a method includes administering to a subject an amount of a nucleic acid encoding a modified blood clotting factor sufficient to decrease clotting time in the subject. In various aspects, the modified blood clotting factor comprises Factor VII, Factor VIII or Factor IX. In additional aspects, the subject is a mammal, e.g., a human.

Methods of reducing the frequency or severity of bleeding in a subject in need of reduced frequency or severity of bleeding also are provided. In one embodiment, method includes administering to a subject an amount of a modified blood clotting factor sufficient to reduce the incidence or severity of a bleeding in the subject. In another embodiment, a method includes administering to a subject an amount of a nucleic acid encoding a modified blood clotting factor sufficient to reduce the incidence or severity of a bleeding in the subject. In various aspects, the modified blood clotting factor comprises Factor VII, Factor VIII or Factor IX. In additional aspects, the subject is a mammal, e.g., a human.

### BRIEF DESCRIPTION OF THE DRAWINGS

FIGS. 1A-1D show three exemplary modified FVII constructs used to generate stable HEK293 cell lines. Short proteolytic cleavage sequences were inserted at position 152 of FVII: (A) wild type FVII; (B) FVII-RKR; (C) FVII-RKRRKR (FVII-2xRKR); and (D) FVII-PRPSRKRR (FVII-INS).

FIGS. 2A-2B show (A) a western blot of expressed wild type and modified FVII proteins from serum free medium of transformed HEK 293 cells and (B) protein gel of purified FVII (4 μg) secreted by HEK 293 cells: 1) protein markers; 2) wild type FVII; 3) FVII-RKR; 4) FVII-2xRKR; 5) FVII-INS; and 6) serum free medium (control). (S) single chain; (H) heavy chain; (L) light chain.

FIGS. 3A-3B show that modified FVII, like wild type FVII, is autoactivated by (A) soluble Tissue factor (sTF) and (B) Factor Xa. −/+symbols indicate the absence and presence of sTF and FXa, respectively.

FIG. 4 shows an exemplary AAV gene delivery vector carrying modified Human Factor VII used for in vivo studes. Expression of FVII is driven by a liver-specific hAAT-ApoE enhancer/promoter construct. ITR: inverted terminal repeat; LSP: liver specific enhancer promoter comprising of 4 ApoE enhancer elements and the promoter of human al antitrypsin (hAAT); Intron: synthetic intron; FVIIa: the cDNA for FVII-2xRKR; hGH polyA: human growth hormone polyadenylation site.

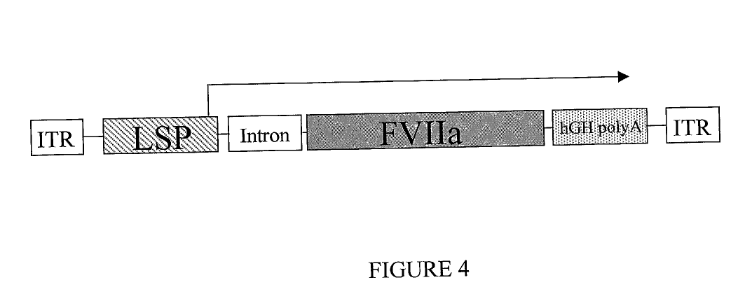

FIGS. 5A-5B show a 2 AAV vector system in which the AAV vector with the modified blood clotting factor FVIIa is under the control of a minimal promoter responsive to tetracycline/doxycycline in the (A) absence and (B) presence of doxycycline (Dox). Symbols are as before.

### DETAILED DESCRIPTION

The present invention is based, at least in part, upon engineering blood clotting factors so that they are produced intracellularly and secreted in their active form. Release from cells of a procoagulent blood clotting factor in its active form into the circulation of an animal initiates or increases blood clotting. For example, Factor VII engineered to have a proteolytic cleavage site not normally present in the factor is cleaved intracellularly at the engineered site to produce a two-chain, activated form, Factor VIIa. Subsequent release of active FVIIa from cells into the circulation of an animal initiates or increases blood clotting. Secretion of a modified blood clotting factor in an active form by cells can result in effective hemostasis of a patient in need of such treatment. Nucleic acids encoding modified blood clotting factors can be introduced into cells of the body using ex vivo or in vivo gene therapy protocols. Modified blood clotting factors and nucleic acids encoding modified blood clotting factors are therefore useful for treating patients having or at risk of having blood clotting deficiencies, such as hemophilia, or other bleeding disorders, including patients who have developed inhibitors.

In accordance with the invention, there are provided modified blood clotting factors and nucleic acid sequences encoding the modified blood clotting factors. In one embodiment, a modified blood clotting factor is engineered to have a proteolytic cleavage site not normally present in the blood clotting factor, where the factor is cleaved at the cleavage site when expressed in an animal cell. In one aspect, a modified blood clotting factor is a vitamin-k dependent procoagulent or anticoagulent protein. In another aspect, a modified blood clotting factor is a functional variant or a functional subsequence of a naturally occurring blood clotting factor (e.g. a factor having increased activity or stability or decreased immunogenicity). In another aspect, a modified blood clotting factor has a mammalian proteolytic cleavage site engineered into it. In yet another aspect, a modified blood clotting factor has a PACE/furin proteolytic cleavage recognition site, or a viral proteolytic cleavage recognition site, or functional variant thereof, engineered into it. In various additional aspects, the modified blood clotting factor is Factor VII, Factor IX, Factor X or protein C.

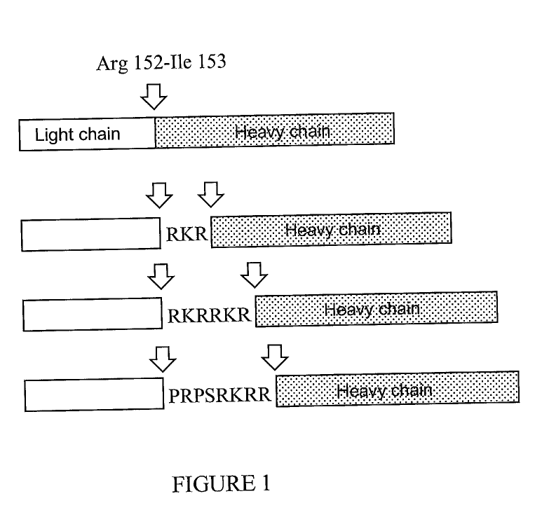

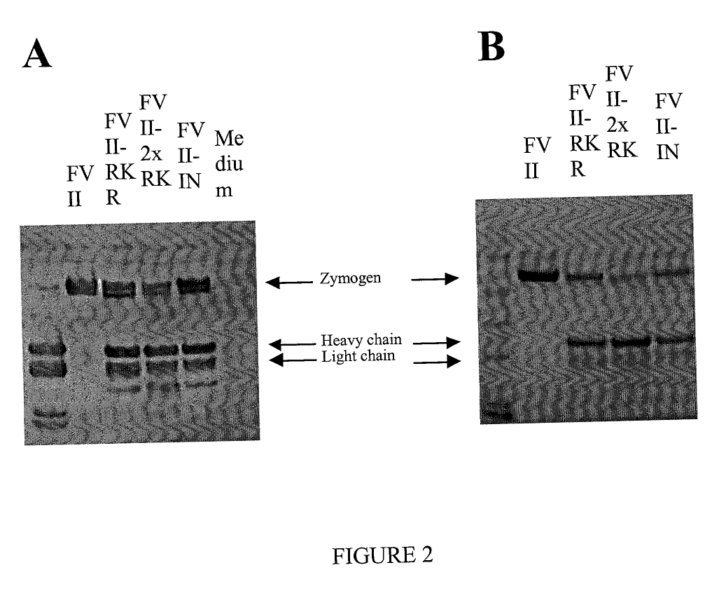

Exemplary modified Factor VII has a protease cleavage site engineered at the normal site of activation (between amino acids Arg¹⁵²-Ile¹⁵³) allowing cleavage of modified Factor VII at the same site as wild type Factor VII. Three different modified FVII constructs were produced, each containing a different cleavage recognition sequence (FIG. 1). Proteolysis of modified Factor VII releases a small peptide and generates a two chain activated Factor VII, Factor VIIa. Expression in HEK 293 cells indicated that cleavage occurred at the correct site to produce Factor VIIa (FIG. 2). N-terminal amino-acid sequencing indicated precise and efficient intracellular cleavage at position Arg152-Ile153, as expected, generating a heavy chain with an identical amino acid sequence to FVIIa.

All three modified FVII constructs were biologically active (60-80% relative to recombinant FVIIa) as assessed by a shortening of the prothrombin time (PT) using human FVII-deficient plasma, as well as by monitoring cleavage of a chromogenic substrate specific for FVIIa (Example 2). A liver hepatoma cell line transduced with an adeno-associated virus, AAV-FVII, expression vector expressed and secreted FVIIa.

In vivo studies in mice were performed using purified FVII wild type protein, one FVIIa modified protein and rFVIIa injected in normal C57BL/6 mice via the tail vein. The injected proteins exhibited half-lives of approximately 30 minutes, similar to the half-life of recombinant FVII. Further in vivo FVIIa expression studies demonstrated that an AAV-FVII expression vector injected into normal immunocompetent or imunodeficient mice could introduce modified FVII into cells and that the transduced cells expressed and secreted protein is the cleaved active form, Factor VIIa (Example 5, see, e.g., Table 2). No inhibitory antibodies against modified FVIIa were detected in these animals.

Blood clotting factors modified in accordance with the invention include any protein component of the clotting cascade, or protein component for which secretion in a cleaved form promotes or initiates clotting or inhibits or decreases clotting. Proteins that promote or initiate clotting are referred to as procoagulent clotting factors. Proteins that inhibit or decrease clotting are referred to as an anticoagulent clotting factors. Proteins that promote or initiate clotting are generally vitamin-K dependent proteins. Specific examples of procoagulent clotting factors include Factor VII, Factor IX and Factor X. Specific examples of anticoagulent clotting factors include activated protein C.

Blood clotting factors that can be modified in accordance with the invention include those of mammalian origin, such as primate (e.g., human, ape, chimpanzee, orangutan, macaque), canine, feline, equine, bovine and porcine, as well as rabbits, rats, mice and guinea pig. The sequences of such proteins as known in the art can be modified in accordance with the invention using routine molecular cloning techniques. Modified factors can be introduced into their corresponding host (e.g., a human modified factor VII introduced into humans), but also can be introduced into a non-corresponding host (e.g., ape modified factor VII introduced into humans, rat into mouse, etc.) in the methods of the invention, so long as the modified factor retains at least partial activity in the administered subject. For example, a non-human primate modified blood clotting factor administered to humans will have at least partial activity of the human blood clotting factor, such as promoting or initiating clotting.

The modified blood clotting factors and nucleic acids encoding the modified blood clotting factors, including variants and subsequences as described herein, can be produced using recombinant nucleic acid cloning and expression methods. For example, nucleic acid encoding a modified blood clotting factor can be produced by recombinant cloning methodologies, inserted into an expression cassette (e.g., vector) and transformed into cells using techniques described herein and further known in the art (Sambrook et al., In: Molecular Cloning: A Laboratory Manual, Cold Spring Harbor Laboratory, ed., 1989). If desired, the modified blood clotting factors may be isolated and purified following transformation of host cells as exemplified herein (immunoaffinity purification, see Example 2) or using other conventional methods known in the art.

Nucleic acid sequences encoding natural blood clotting factors are known in the art or can be isolated from organisms in which they are expressed and used as templates for constructing the nucleic acids encoding the modified blood clotting factors in accordance with the invention. Such techniques therefore include, but are not limited to: 1) hybridization of genomic or cDNA libraries with probes to detect homologous nucleic acid sequences which can then be cloned into a plasmid, propagated amplified and purified; 2) antibody screening to detect polypeptides having shared structural features, for example, using an expression library; 3) amplification, e.g., polymerase chain reaction (PCR), with genomic DNA or cDNA targets using primers (e.g., a degenerate primer mixture) capable of annealing to a nucleotide encoding a blood clotting factor; and 4) computer searches of sequence databases for related sequences.

The modified blood clotting factors encoded by nucleic acids include full-length native sequences, as with naturally occurring proteins, as well as variant forms or and subsequences. The invention therefore includes variant modified blood clotting factors having one or more amino acid substitutions or additions relative to a comparison (e.g., wild type) sequence, referred to herein as “variants,” as well as subsequences that are at least one amino acid less in length than the comparison full length sequence.

Variants and subsequences refer to insertions, additions, substitutions and deletions of the invention modified clotting factors and nucleic acids encoding the modified blood clotting factors. An “insertion” or “addition” means an internal or terminal addition of one or more amino acid or nucleotide residues, respectively, as compared to the comparison molecule. A “substitution” means the replacement of one or more amino acids or nucleotides by different amino acids or nucleotides, respectively. A “deletion” means a change in either amino acid or nucleotide sequence in which one or more amino acid or nucleotide residues, respectively, are absent compared to the comparison molecule.

Variants include functional forms, meaning that the variant form has at least one of the functions or activities of an unmodified or comparison sequence, referred to herein as “functional variants” or “active variants.” Functional variants need only have one of the functions or activities of an unmodified or comparison sequence, but can have additional functions of the unmodified or comparison sequence (two or more).

Functional variants also include modified blood clotting factors combined with one more functionality's distinct from the unmodified or comparison sequence, e.g., a chimeric or fusion polypeptide comprising the variant sequence and a second amino acid sequence conferring a distinct functionality, such as a protein tag (e.g., T7, GCT, polyhistidine, immunoglobulin, etc.). Accordingly, invention modified blood clotting factors further include amino acid sequence additions having a distinct functionality or activity.

Functional variants need only have at least part of the given function or activity of an unmodified or comparison sequence. For example, a functional variant of a wild type Factor VIIa may have less activity than wild type Factor VIIa. The activity may be as little as 10-20% of wild type activity up to 100% of wild type activity. Functional variants can have greater function or activity than an unmodified or comparison sequence. For example, a functional variant of Factor VIIa may have greater activity than wild type Factor VIIa. The activity may be 110-150% of wild type activity up to 200% or more of wild type activity.

Activities or functions of the variants included are the activities and functions associated with the procoagulent and anticoagulent factors as set forth herein or otherwise known in the art. For example, Factor VIIa initiates the clotting cascade by complexing with tissue factor. The FVIIa/TF complex then cleaves FIX and FX to produce FIXa and FXa, respectively. FXa activates prothrombin to thrombin and protein C to activated protein C. Thrombin eventually gives rise to a fibrin clot whereas activated protein C is an anticoagulent. Thus, activities or functions of FVII include autoactivation when associated with TF all the way through the various steps of the clotting cascade, e.g., activation of FIX to FIXa and FX to FXa, etc.

Functional variants of the invention include substitutions and additions to the amino acid sequence of the modified blood clotting factor that increase or improve therapeutic efficacy. For example, increasing activity of a factor (e.g., the amount needed to initiate or promote blood clotting) in a subject can decrease the amount of factor needed to restore hemostasis in a subject by having a more active protein in the circulation. A variant Factor VII having greater activity than wild type Factor VII is described, for example, in Shah et al., Proc. Natl. Acad. Sci. USA 95:4229 (1998).

Increasing stability of a modified blood clotting factor in a subject can increase the circulating half-life of the protein thereby prolonging the protein's activity in the body. Decreasing immunogenicity of a modified blood clotting factor can also prolong activity by reducing the amount or affinity of inhibitory antibodies that bind to the clotting factor which in turn reduce or prevent a function or activity of the factor.

“Functional subsequences” mean a subsequence that retains one or more functions or activities of the comparison sequence, i.e., the longer sequence of which the subsequence is based upon. As with variant sequences, functional subsequences need only have at least part of the given function or activity of a comparison sequence. Thus, a functional subsequence of a wild type Factor VIIa may have less activity than wild type Factor VIIa. The activity may be as little as 10-20% of wild type activity up to 100% of wild type activity. Functional subsequences can have greater function or activity than a comparison sequence, e.g., the activity may be 110-150% of wild type activity up to 200% or more of wild type activity.

Routine assays known in the art can be used to identify functional variants and subsequences having one or more functions or activities of wild type factor. For example, to identify a functional variant or subsequence of a procoagulent clotting factor, prothrombin conversion time (PT) using plasma deficient in the test factor can be measured. Another assay is to measure cleavage of a chromogenic substrate specific for the test factor (see, e.g., Example 2). For example, conversion of Factor X to Factor Xa can be measured in the presence of a Factor VIIa variant or subsequence. To determine stability in the circulation, the test factor can be injected into animals and their half-measured. Restoration of blood clotting, at least in part, in an animal model, such as a hemophilac animal, is also an assay for identifying functional variants and subsequences.

The modified blood clotting factors of the invention can be engineered to include any proteolytic cleavage site recognized by an intracellular protease so that the secreted protein has been cleaved. Amino acid sequences recognized by intracellular proteases located in the endoplasmic reticulum-golgi transport pathway are known in the art and include PACE/furin sites. In addition, stretches of basic amino residues are known to be cleaved by intracellular proteases. Other proteolytic cleavage sites include those present on virus proteins, which often utilize cellular proteases for processing. For example, retroviral envelope and gag proteins are cleaved by intracellular proteases and the cleavage/recognition sequences in these proteins can be used in producing the modified blood clotting factors of the invention. Exemplary proteolytic cleavage recognition sites are RKR, RKRRKR (SEQ ID NO:1) and PRPSRKRR (SEQ ID NO:2), which is derived from the C-terminus of the a-chain of the human insulin receptor. Additional protein cleavage/recognition sites can be identified by sequencing the site of cleavage on a cleaved/secreted protein and determining whether recombinantly introducing the site into a different protein targeted for secretion mediates cleavage of the protein at the site.

The position of the engineered proteolytic cleavage site within the modified blood clotting factors of the invention will likely be the same as the natural wild type (native) proteolytic cleavage site. That is, the engineered cleavage/recognition site will substitute for the native cleavage/recognition site. The engineered cleavage/recognition site need not have the same amino acid length as the native cleavage/recognition site, so long as the modified clotting factor has one or more activities or functions of wild type clotting factor. In other words, the engineered cleavage/recognition site may be longer or shorter than the native site, resulting in the addition or deletion of amino acids to the clotting factor in comparison to the wild type factor.

The position of the proteolytic cleavage site also need not be identical to that of the native site so long as the cleaved modified clotting factor has one or more activities or functions as the wild type clotting factor. Thus, the position of the engineered proteolytic cleavage site within a modified clotting factor of the invention may vary relative to the position of the native site from about 25-50 amino acids, or less. In other words, the engineered proteolytic cleavage site can be introduced by an in-frame insertion into the clotting factor so that it is within about 25-50 amino acids, or less, from the native site. For such modified factors, the native site may be mutated/removed so that it is no longer functional or, alternatively, need not be mutated/removed, in which case there are two cleavage recognition sites in the modified blood clotting factor, the engineered proteolytic cleavage site and the native site.

Nucleic acid sequences encoding modified blood clotting factors typically include an expression control element in order to confer expression of the factor in vitro or in vivo. Thus, the invention provides nucleic acids sequences encoding modified blood clotting factors operatively linked to an expression control element. In one embodiment, the control element is a regulatable or tissue specific expression control element, e.g., a regulatable or tissue specific promoter. In one aspect, the promoter comprises a skeletal muscle actin promoter or a muscle creatine kinase promoter. In another embodiment, the tissue-specific expression control element confers expression of the modified blood clotting factor in muscle, liver, kidney or blood vessel endothelium. In another aspect, the regulatable expression control element comprises elongation factor 1α promoter.

The term “expression control element” refers to one or more nucleotide sequences that influence expression of a nucleic acid to which it is in operatively linked. The term “operatively linked” refers to a physical or a functional relationship between the control element and the nucleic acid such that the control element modulates transcription and as appropriate, translation of the transcript. Expression control elements therefore include promoters, enhancers, transcription terminators, a start codon (e.g., ATG), etc.

Expression control elements include elements that activate transcription constituitively, elements that are inducible (i.e., require a signal for activation), and elements that are derepressible (i.e., require a signal for inactivation; when the signal is absent, transcription is activated or “derepressed”). Also included are elements sufficient to render gene expression controllable at particular cell differentiation stages, for specific cell-types, tissues or physiological conditions. Typically, control elements are located upstream or downstream (i.e., 5′ and 3′) of the coding sequence, but can be present within the gene, such as within introns. Promoters and enhancers are generally located 5′ of the coding sequence, although enhancers can function to control expression when located 3′ of the nucleic acid, and at significant distances from the nucleic acid. A “promoter” is meant a minimal nucleic acid sequence element sufficient to initiate transcription. Promoters, enhancers and the like produced by recombinant DNA or synthetic techniques can be used to provide for transcription of the nucleic acids encoding modified blood clotting factors.

Expression control elements need not be physically linked to the nucleic acid in order to control expression. For example, a minimal element can be linked to a nucleic acid encoding a modified clotting factor on a first vector. A second vector can contain a second element that controls expression of an operatively linked nucleic acid encoding a protein that binds to the minimal element on the first plasmid, thereby influencing expression of the modified blood clotting factor. Because the second element that regulates expression of the protein in turn regulates expression of the modified blood clotting factor, the second element is operatively linked to the nucleic acid encoding the modified blood clotting factor.

Thus, as an alternative, expression factors can be controlled by operatively linking nucleic acid encoding a modified blood clotting to an expression control element that is responsive to a drug or other small molecule, that is generally inactive in the absence of the drug or small molecule so that the drug will specifically upregulate expression of the clotting factor transgene. Such a strategy provides a more refined level of in vivo modified clotting factor expression control. In this way, the level of expression can be modulated by the amount of drug administered to the subject.

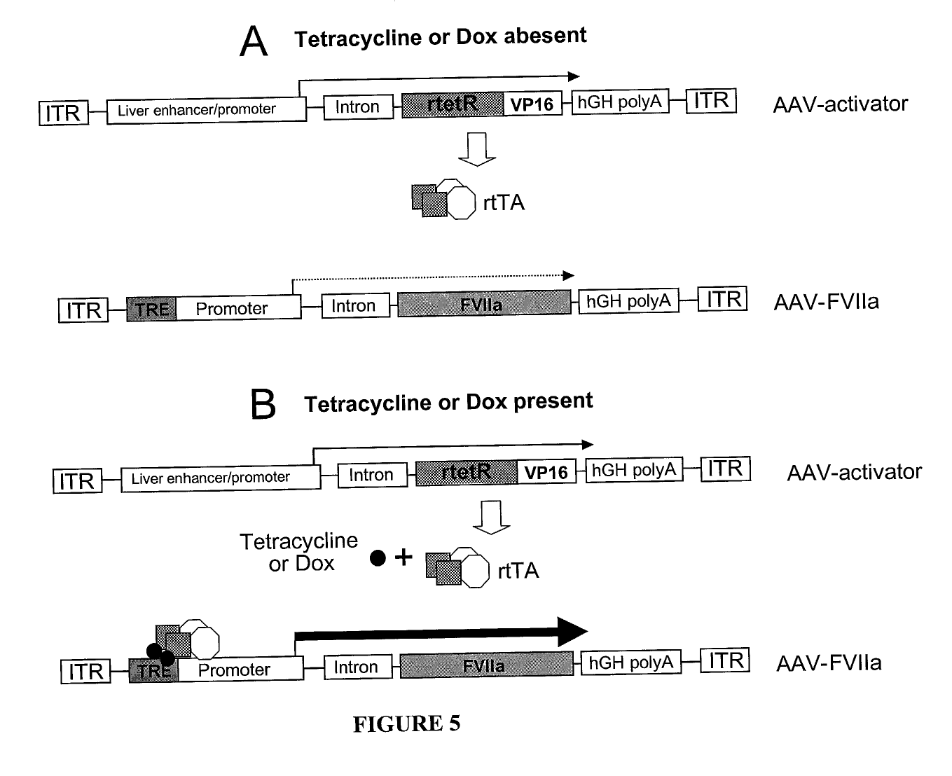

For example, using a two AAV vector approach, a first AAV vector with the modified blood clotting factor transgene (e.g., FVIIa) under the control of a minimal promoter can be responsive to tetracycline (or an analogue such as doxycycline) via a tetracycline response element. The tetracycline responsiveness will be provided by a second AAV vector co-injected with the first; the second vector will encode a protein that upon binding to tetracycline binds to the tetracycline response element on the first FVIIa AAV vector, and will therefore increase expression of FVIIa (see, e.g., FIG. 5). This approach has been shown to increase gene expression up to 2-3 orders of magnitude, reaching maximal levels at 24 h after drug administration (Kistner, et al., Proc. Nat. Acad. Sci. USA 93:10933 (1996)). Such a system will be useful to more precisely modulate levels of modified blood clotting factor expression to maximize safety in treatment of patients having or at risk of having a bleeding or clotting disorder.

Thus, for expression in cells, invention nucleic acids may be inserted into a vector. The term “vector,” e.g., a plasmid, virus or other vehicle known in the art can be manipulated by insertion or incorporation of a nucleic acid for genetic manipulation (i.e., “cloning vectors”) or can be used to transcribe or translate the inserted nucleic acid (i.e., “expression vectors”). Such vectors are therefore useful for producing the nucleic acids encoding the modified blood clotting factors and expressing the encoded modified blood clotting factors, variants and subsequences described herein.

A vector generally contains at least an origin of replication for propagation in bacteria or eukaryotic cells and a promoter. Control elements, including expression control elements as set forth herein, present within a vector are included to facilitate transcription and translation. “Control Elements” therefore include, at a minimum, one or more components whose presence can influence expression, that is, increase or decrease expression. Control elements also can include splicing signal for introns, maintenance of the correct reading frame of the gene to permit in-frame translation of mRNA, stop codons, a polyadenylation signal, leader sequences and fusion partner sequences.

For expression or cloning in bacterial systems, constitutive promoters such as T7 and the like, as well as inducible promoters such as pL of bacteriophage λ, plac, ptrp, ptac (ptrp-lac hybrid promoter) may be used. When expressing in insect cell systems, constitutive or inducible promoters (e.g., ecdysone) may be used. In yeast, vectors containing constitutive or inducible promoters may be used (see, e.g., Ausubel et al., In: Current Protocols in Molecular Biology, Vol. 2, Ch. 13, ed., Greene Publish. Assoc. & Wiley Interscience, 1988; Grant et al., (1987) In: Methods in Enzymology, 153, 516-544, eds. Wu & Grossman, 31987, Acad. Press, N.Y.; Glover, DNA Cloning, Vol. 11, Ch. 3, IRL Press, Wash., D.C., 1986; Bitter (1987) In: Methods in Enzymology, 152, 673-684, eds. Berger & Kimmel, Acad. Press, N.Y.; and, Strathem et al., The Molecular Biology of the Yeast Saccharomyces (1982) eds. Cold Spring Harbor Press, Vols. I and II). A constitutive yeast promoter such as ADH or LEU2 or an inducible promoter such as GAL may be used (R. Rothstein In: DNA Cloning, A Practical Approach, Vol. 11, Ch. 3, ed. D. M. Glover, IRL Press, Wash., D.C., 1986).

When expressing in mammalian cell systems, constitutive promoters of viral or other origins may be used. For example, SV40, or viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; the inducible mouse mammary tumor virus LTR) can be used for expression.

For long-term expression, stable expression is preferred. Although the invention is not bound or limited by any particular theory, stable maintenance of expression vectors in mammalian cells is believed to occur by integration of the vector into a chromosome of the transformed cell. The expression vector also can contain a nucleic acid encoding a selectable marker (e.g., neo and hygromycin genes) conferring resistance to a selective pressure or an identifiable marker (e.g., β-galactosidase), thereby allowing cells having the vector to be identified, grown and expanded. Alternatively, a selectable marker can be on a second vector, which is cotransfected into a host cell with a first vector containing an invention polynucleotide.

Additional selection systems may be used, including, but not limited to the herpes simplex virus thymidine kinase gene (Wigler et al., Cell 11:223 (1977)), hypoxanthine-guanine phosphoribosyltransferase gene (Szybalska et al., Proc. Natl. Acad. Sci. USA 48:2026 (1962)), and the adenine phosphoribosyltransferase (Lowy et al., Cell 22:817 (1980)) genes can be employed in tk−, hgprt− or aprt− cells respectively. Additionally, antimetabolite resistance can be used as the basis of selection for dhfr, which confers resistance to methotrexate (Wigler et al., Proc. Natl. Acad. Sci. USA 77:3567 (1980); O'Hare et al., Proc. Natl. Acad. Sci. USA 78:1527 (1981)); the gpt gene, which confers resistance to mycophenolic acid (Mulligan et al., Proc. Natl. Acad. Sci. USA 78:2072 (1981)); the neomycin resistance gene, which confers resistance to the aminoglycoside G-418 (Colberre-Garapin et al., J. Mol. Biol. 150:1 (1981)); the zeocin resistance gene (Stratagene), and the hygromycin resistance gene, which confers resistance to hygromycin (Santerre et al., Gene 30:147 (1984)). Additional selectable genes have been described, namely trpB, which allows cells to utilize indole in place of tryptophan; hisD, which allows cells to utilize histinol in place of histidine (Hartman et al., Proc. Natl. Acad. Sci. USA4 85:8047 (1988)); and ODC (ornithine decarboxylase), which confers resistance to the ornithine decarboxylase inhibitor, 2-(difluoromethyl)-DL-ornithine, DFMO (McConlogue (1987) In: Current Communications in Molecular Biology, Cold Spring Harbor Laboratory, ed.).

In yeast, vectors that facilitate integration of foreign nucleic acid sequences into a chromosome, via homologous recombination, for example, are known in the art and can be used. Yeast artificial chromosomes (YAC) are typically used when the inserted nucleic acids are too large for more conventional vectors (e.g., greater than about 12 kb).

Mammalian expression systems further include vectors specifically designed for in vivo and ex vivo expression. Such systems include adeno-associated virus vectors (U.S. Pat. No. 5,604,090;). AAV vectors have previously been shown to provide expression of Factor IX in humans and in mice at levels sufficient for therapeutic benefit (Kay et al., Nat. Genet. 24:257 (2000); Nakai et al., Blood 91:4600 (1998)). Adenoviral vectors (U.S. Pat. Nos. 5,700,470, 5,731,172 and 5,928,944), herpes simplex virus vectors (U.S. Pat. No. 5,501,979) and retroviral (e.g., lentivirus vectors are useful for infecting dividing as well as non-dividing cells and foamy virues) vectors (U.S. Pat. Nos. 5,624,820, 5,693,508, 5,665,577, 6,013,516 and 5,674,703 and WIPO publications WO92/05266 and WO92/14829) and papilloma virus vectors (e.g., human and bovine papilloma virus) have all been employed in gene therapy (U.S. Pat. No. 5,719,054). Vectors also include cytomegalovirus (CMV) based vectors (U.S. Pat. No. 5,561,063). Vectors that efficiently deliver genes to cells of the intestinal tract have been developed and also may be used (see, e.g., U.S. Pat. Nos. 5,821,235, 5,786,340 and 6,110,456). Additional viral vectors useful for expression include parvovirus, rotavirus, Norwalk virus, coronaviruses, paramyxo and rhabdoviruses, togavirus (e.g., sindbis virus and semliki forest virus) and vesicular stomatitis virus.

Introduction of nucleic acid and polypeptide in vitro, ex vivo and in vivo can also be accomplished using other techniques. For example, an expression control element in operable linkage with a nucleic acid encoding a modified blood clotting factor can be incorporated into particles or a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid drug delivery system. Colloidal dispersion systems include macromolecule complexes, nano-capsules, microspheres, beads, and lipid-based systems, including oil-in-water emulsions, micelles, mixed micelles, and liposomes.

The use of liposomes for introducing various compositions, including nucleic acids, is known to those skilled in the art (see, e.g., U.S. Pat. Nos. 4,844,904, 5,000,959, 4,863,740, and 4,975,282). A carrier comprising a natural polymer, or a derivative or a hydrolysate of a natural polymer, described in WO 94/20078 and U.S. Pat. No. 6,096,291, is suitable for mucosal delivery of molecules, such as polypeptides and polynucleotides. Piperazine based amphilic cationic lipids useful for gene therapy also are known (see, e.g., U.S. Pat. No. 5,861,397). Cationic lipid systems also are known (see, e.g., U.S. Pat. No. 5,459,127). Accordingly, viral and non-viral vector e.g., plasmid DNA, means of delivery into cells or tissue, in vitro, in vivo and ex vivo are included.

In instances where cell or tissue targeting is desired, an invention composition can of course be delivered to the target organ or tissue by injection or infusion or the like. Targeting can also be achieved by using proteins that bind to a cell surface protein (e.g., receptor or matrix protein) present on the cell or population of cell types. For example, antibodies or antibody subsequences (e.g., Fab region) that bind to a cell surface protein can be included in the delivery systems in order to facilitate cell or tissue targeting. Viral coat proteins that bind particular cell surface proteins can be used to target cells or tissues for expression of the modified blood clotting factors of the invention. For example, naturally occurring or synthetic (e.g. recombinant) retroviral envelope proteins with known cell surface protein binding specificity can be employed in the retroviral vectors or liposomes containing nucleic acid encoding a modified blood clotting factor in order to intracytoplasmically deliver the factor into target cells expressing the cell surface protein. Thus, delivery vehicles, including viral vectors and colloidal dispersion systems, can be made to have a coat protein or a proteinaceous coat in order to facilitate targeting or intracytoplasmic delivery and expression of a modified blood clotting factor.

The invention therefore also provides transformed cells and progeny thereof into which a nucleic acid molecule of the invention has been introduced by means of recombinant DNA techniques. The transformed cells can be propagated and the introduced nucleic acid transcribed, or encoded protein expressed. It is understood that progeny may not be identical to the parental cell, since there may be mutations that occur during replication. Transformed cells include but are not limited to bacteria, fungi, plant, insect, parasites and animal (mammalian, including human) cells.

The term “transformed” means a genetic change in a cell following incorporation of nucleic acid (e.g., a transgene) exogenous to the cell. Thus, a “transformed cell” is a cell into which, or a progeny of which a nucleic acid molecule has been introduced by means of recombinant techniques. Cell transformation with nucleic acid in order to produce host cells may be carried out as described herein or otherwise using techniques known in the art. Accordingly, methods of producing cells containing the nucleic acids and expressing the modified blood clotting factor of the invention are also provided.

Transformed cells also include cells present in the body, including cells of a tissue, whether normal or aberrant (e.g., diseased). Transformed cells therefore include, for example, liver cells, muscle cells, including stem cells, multipotent or pluripotent progenitor cells, bone marrow, heart, larynx, lung, spleen, pancreas, bladder, gastrointestinal tract cells (mouth, tongue, buccal tissue, esophagus, stomach, small intestine, large intestine and rectum), skin, in short, cells of all lineages and differentiation states, in a subject or in a tissue or organ of a subject.

The invention provides kits comprising invention compositions, including pharmaceutical formulations, packaged into in suitable packaging material. In one embodiment, a kit includes a modified blood clotting factor. In another embodiment, a kit includes a nucleic acid encoding a modified blood clotting factor. In additional embodiments, the nucleic acids further include an expression control element conferring expression in a cell; an expression vector; a viral expression vector; an adeno-associated virus expression vector; an adenoviral expression vector; and a retroviral expression vector. In yet other embodiments, a modified blood clotting factor or a nucleic acid encoding a modified blood clotting factor is included in a colloidal dispersion system; a liposome; a cationic liposome; and an anionic liposome. In additional embodiments, a kit includes a label or packaging insert including instructions for expressing a modified blood clotting factor or a nucleic acid encoding a modified blood clotting factor in cells in vitro, in vivo, or ex vivo.

As used herein, the term “packaging material” refers to a physical structure housing the components of the kit, such as modified blood clotting factor or a nucleic acid encoding a modified blood clotting factor. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention, e.g., treating a bleeding or clotting disorder.

Kits of the invention therefore can additionally include instructions for using the kit components in a method of the invention. Instructions can include instructions for practicing any of the methods of the invention described herein. Thus, the pharmaceutical compositions can be included in a container, pack, or dispenser together with instructions for administration. Instructions may additionally include indications of a satisfactory clinical endpoint or any adverse symptoms that may occur, or additional information required by the Food and Drug Administration for use on a human.

The instructions may be on “printed matter,” e.g., on paper of cardboard within the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Invention kits can additionally include a buffering agent, a preservative, or a protein/nucleic acid stabilizing agent. The kit can also include components for assaying for blood clotting time of a sample from a subject, for example to test clotting time before and after administering a modified blood clotting factor to the subject. The kit can also contain a control sample or a modified clotting factor to be used as standard. Each component of the kit can be enclosed within an individual container and all of the various containers can be within a single package.

The nucleic acids encoding a modified blood clotting factor, including an expression control element in operable linkage therewith, can be introduced for stable expression into cells of a whole organism. Such organisms include non-human transgenic animals, which are useful for studying the effect of a modified blood clotting factor in the animal and therapeutic benefit. For example, as described herein, expression of a modified blood clotting factor in a mouse having a blood clotting disorder (e.g., a hemophilac mouse) can protect the animal from bleeding episodes. Mice strains that have bleeding disorders are particularly attractive targets for making transgenic mice that express a modified blood clotting factor of the invention in order to study the effect of protein expression in the afflicted mouse. Transgenic and genetic animals that are susceptible to bleeding disorders, including hemophilac dogs, are known in the art and are appropriate targets for expressing a modified blood clotting factor.

The term “transgenic animal” refers to an animal whose somatic or germ line cells bear genetic information received, directly or indirectly, by deliberate genetic manipulation at the subcellular level, such as by microinjection or infection with recombinant virus. The term “transgenic” further includes cells or tissues (i.e., “transgenic cell,” “transgenic tissue”) obtained from a transgenic animal genetically manipulated as described herein. In the present context, a “transgenic animal” does not encompass animals produced by classical crossbreeding or in vitro fertilization, but rather animals in which one or more cells receive a nucleic acid molecule. Invention transgenic animals can be either heterozygous or homozygous with respect to the transgene. Methods for producing transgenic animals, including mice, sheep, pigs and frogs, are well known in the art (see, e.g., U.S. Pat. Nos. 5,721,367, 5,695,977, 5,650,298, and 5,614,396) and, as such, are additionally included.

Thus, in accordance with the invention, there are provided non-human transgenic animals that produce a modified blood clotting factor, production not naturally occurring in the animal, production conferred by a transgene present in somatic or germ cells of the animal. In one embodiment, the transgene comprises a nucleic acid, including an expression control element in operable linkage with a nucleic acid encoding a modified blood clotting factor (e.g., Factor VII, Factor X or protein C, among others). In one aspect, the transgenic animal is a mouse.

The modified blood clotting factors of the invention, including variants and subsequences thereof and nucleic acids encoding the modified blood clotting factors, variants and subsequences thereof, can be incorporated into pharmaceutical compositions. Such pharmaceutical compositions are useful for administration to a subject in vivo or ex vivo, and for providing therapy for a clotting or bleeding disorder to practice the methods of the invention, for example.

Pharmaceutical compositions include “pharmaceutically acceptable” and “physiologically acceptable” carriers, diluents or excipients. As used herein the term “pharmaceutically acceptable” and “physiologically acceptable” includes solvents (aqueous or non-aqueous), solutions, emulsions, dispersion media, coatings, isotonic and absorption promoting or delaying agents, compatible with pharmaceutical administration. Such formulations can be contained in a tablet (coated or uncoated), capsule (hard or soft), microbead, emulsion, powder, granule, crystal, suspension, syrup or elixir. Supplementary active compounds (e.g., preservatives, antibacterial, antiviral and antifungal agents) can also be incorporated into the compositions.

Pharmaceutical compositions can be formulated to be compatible with a particular route of administration. Thus, pharmaceutical compositions include carriers, diluents, or excipients suitable for administration by various routes.

Pharmaceutical compositions for parenteral, intradermal, or subcutaneous administration can include a sterile diluent, such as water, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic.

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. In many cases, isotonic agents, for example, sugars, polyalcohols such as manitol, sorbitol, sodium chloride are included in the composition. Including an agent which delays absorption, for example, aluminum monostearate and gelatin can prolonged absorption of injectable compositions.

Sterile injectable solutions can be prepared by incorporating the active compound in the required amount in an appropriate solvent with one or a combination of above ingredients followed by filtered sterilization. Generally, dispersions are prepared by incorporating the active compound into a sterile vehicle containing a basic dispersion medium and other ingredients from those above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. For transdermal administration, the active compounds are formulated into ointments, salves, gels, or creams as generally known in the art.

Invention modified blood clotting factors, variants and subsequences, and nucleic acids encoding the modified blood clotting factors, variants and subsequences can be prepared with carriers that will protect them against rapid elimination from the body, such as a controlled release formulation or a time delay material such as glyceryl monostearate or glyceryl stearate. The compositions can also be delivered using implants and microencapsulated delivery systems.

Biodegradable, biocompatable polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to cells or tissues using antibodies or viral coat proteins) can also be used as pharmaceutically acceptable carriers. These can be prepared according to methods known to those skilled in the art, for example, as described in U.S. Pat. No. 4,522,811.

A pharmaceutical composition including a gene therapy vector can include the gene therapy vector in an acceptable excipient, diluent or carrier, or can comprise a slow release matrix in which the gene delivery vehicle is imbedded. Alternatively, where the gene delivery vector is produced intact from recombinant cells, e.g., retroviral vectors, the pharmaceutical composition can include one or more of the cells that produce the gene delivery vector.

Additional pharmaceutical formulations appropriate for administration are known in the art and are applicable in the methods and compositions of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993)).

The pharmaceutical formulations can be packaged in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the pharmaceutical carrier or excipient.

Invention modified blood clotting factors can promote or initiate clotting or can inhibit or reduce clotting in a subject. The invention compositions are therefore useful for treating a subject having or at risk of having a clotting disorder. A subject may have or be at risk of having insufficient clotting or excessive clotting. For example, a subject may have insufficient amounts or activity of one or more procoagulent clotting factors so that clotting time is longer than normal resulting in a bleeding or clotting disorder. A subject may also have a defect in platelets causing reduced aggregation to form a clot resulting in a bleeding or clotting disorder.

Thus, in accordance with the invention there are provided methods for treating a bleeding or clotting disorder of a subject having or at risk of having a bleeding or clotting disorder. In one embodiment, a method includes administering to a subject having or at risk of having a bleeding or clotting disorder an amount of a modified blood clotting factor sufficient to reduce one or more symptoms of the disorder. In one aspect, the disorder is amenable to treatment with Factor VII, Factor VIII or Factor IX. In another aspect, the disorder is caused by insufficient activity or expression of a vitamin-K dependent procoagulent. In yet another aspect, the disorder is caused by insufficient platelet aggregation. In still another aspect, the disorder comprises hemophilia (e.g., hemophilia A or hemophilia B) or Factor VII deficiency. In particular aspects, the disorder comprises Glanzmann's or Bernard-Soulier's thrombasthenia.

Target subjects include those having a bleeding or clotting disorder caused by insufficient or deficient expression or activity of a clotting factor. Specific examples include hemophiliacs, such as hemophilia A and B. Hemophiliacs are identified according to the amount of Factor VIII or Factor IX. Normal levels for factor VIII are 100-200 ng/ml and for Factor IX, 5 μg/ml. Patients with less than 1% of these levels are classified as severe, those with 1-5% are classified as moderate and those with greater than 5% are classified as mildly affected. Severe, moderate and mildly affected hemophiliacs are appropriate targets.

Target subjects also include those having a bleeding or clotting disorder caused by a defect in platelets leading to insufficient or deficient aggregation in clot formation. Glanzmann's thrombasthenia and Bernard-Soulier's thrombasthenia are specific examples of disorders caused by defects in platelets.

Target subjects include those who produce inhibitors, such as inhibitory antibodies, to any blood clotting factor. For example, as a consequence of repeated administration of clotting factor preparations, hemophilic patients can develop inhibitory antibodies against blood clotting factors, such as factor VIII or factor IX. Such subjects, who have inhibitory antibodies or other inhibitors of blood clotting factor expression, function or activity, are amenable to treatment with the modified blood clotting factors of the invention.

The invention methods are therefore also applicable to treating subjects who have developed inhibitors of a blood clotting factor such as inhibitory antibodies that bind to and inhibit activity of a procoagulent factor. Thus, in additional aspects, a subject treated in a method of the invention produces an inhibitor (e.g., inhibitory antibodies) to a blood clotting factor (e.g., Factor VIII or Factor IX).

Target subjects also include those having a longer clotting time in comparison to what is considered physiologically normal (in comparison to matched controls, e.g. age, race, gender matched). Such target subjects may not exhibit overt symptoms of a bleeding or clotting disorder but are nevertheless appropriate for practicing the methods of the invention.

Thus, in accordance with the invention, there are also provided methods for decreasing clotting time in a subject in need of decreased clotting time. In one embodiment, a method includes administering to the subject an amount of a modified blood clotting factor sufficient to decrease clotting time in the subject. In one aspect, the modified blood clotting factor comprises Factor VII, Factor VIII or Factor IX. In another aspect, the subject is a mammal (e.g., human).

Target subjects further include subjects having increased frequency or severity of bleeding episodes in comparison to what is considered physiologically normal. Frequency means the number of bleeding episodes. Severity means the amount of bleeding that occurs, internally (e.g., hemorrhage) or externally.

Thus, in accordance with the invention, there are also provided methods for reducing the frequency or severity of bleeding in a subject. In one embodiment, a method includes administering to the subject an amount of a modified blood clotting factor sufficient to reduce the incidence or severity of bleeding in the subject. In one aspect, the modified blood clotting factor comprises Factor VII, Factor VIII or Factor IX. In another aspect, the subject is a mammal (e.g., human).

Target subjects may be treated after symptoms of the bleeding or clotting disorder manifest or prior to their manifestation. Thus, prophylactic treatment methods also are included.

Target subjects include those at risk of developing a bleeding or clotting disorder, such as a subject who lacks or is deficient in an intrinsic coagulation pathway protein, has inhibitors to these proteins, or has some other hemostatic abnormality which would benefit by administration of a modified blood clotting factor (e.g., Factor VIIa). Subjects appropriate for treatment additionally include those having a genetic predisposition or family history to developing a bleeding or clotting disorder. For example, subjects which have a genetic lesion (mutation or deletion) in a blood clotting factor (e.g., Factor IX) or other protein of the cascade are candidate target subjects because administering a modified blood clotting factor of the invention to such subjects can prevent or delay development of symptoms, or reduce the severity of symptoms caused by the lesion. Subjects at risk of developing a clotting or bleeding disorder can be identified using routine genetic screening for the presence of the genetic lesion or inquiry into the subjects' family history to establish that they are at risk of the disorder.

Target subjects may be deficient in expression or activity of a particular kind of coagulation pathway protein but can be treated with a different blood clotting factor modified in accordance with the invention. For example, a modified Factor VII (which is cleaved to produce Factor VIIa) can be introduced into a subject who has deficient or insufficient expression or activity of Factor VII as well as Factors VIII or IX. Thus, the treatment methods of the invention include treating a subject with has deficient or insufficient expression or activity of a particular blood clotting factor with a different blood clotting factor modified in accordance with the invention.

The term “subject” refers to animals, typically mammalian animals, such as a non human primate (apes, gibbons, chimpanzees, orangutans, macaques), a domestic animal (dogs and cats), a farm animal (horses, cows, goats, sheep, pigs), experimental animal (mouse, rat, rabbit, guinea pig) and humans. Subjects include disease model animals (e.g., hemophilic animals, such as mice and dogs).

In the methods of the invention, including prophylactic and therapeutic treatments, the methods may be specifically tailored or modified, based on pharmacogenomic data. As used herein, “pharmacogenomics” refers to the application of genomics technology such as gene sequencing, statistical genetics, and expression analysis to drugs. The term refers to characterizing how a patient's genes determine their response to a drug (e.g., a patient's “drug response phenotype or genotype”.) Pharmacogenomics therefore allows a clinician to target prophylactic or therapeutic treatments to patients who are likely to benefit from the treatment while avoiding treating patients whose response genotype or phenotype is indicative of an adverse side effect. Thus, the prophylactic or therapeutic methods of the invention can be tailored based upon an individual's drug response genotype.

Target cells and tissues for the practicing the methods of the invention can be any cell or tissue capable of releasing, directly or indirectly, the cleaved form of the modified blood clotting factor into the circulation. Likely targets are cells/tissues known to produce blood clotting factors in vivo, such as the liver. Other targets are cells/tissue that produce proteins present in the circulatory system including, for example, muscle, spleen, pancreas, heart, lung, blood vessel cells (e.g., endothelium), gut (e.g., mouth, esophagus, stomach, small intestine and large intestine), and hematopoeitic cells (e.g., bone marrow). Additional cells/tissue that can be targeted include kidney. Thus, essentially any vascularized tissue or organ can be targeted for expressing the modified blood clotting factors of the invention.

Liver and muscle are also attractive targets because both have been shown to express transgenes at high levels. For example, injection of an AAV vector with a human factor IX transgene under the control of the elongation factor 1α promoter in the portal vein of mice resulted in levels of 0.7 to 3.3 μg/mL of factor IX in the circulation (Nakai et al., Blood 91:4600 (1998)). These levels of gene product are in the therapeutic range for activated factor VII (FVIIa) in a gene therapy setting.

The modified blood clotting factors can therefore be administered systemically to a subject or by targeted delivery to a cell, tissue or organ of a subject, by any appropriate route. Particular examples of routes of administration include parenteral, e.g., intravenous, intrarterial, intramuscular, intradermal, subcutaneous, intracranial, oral (e.g., inhalation), transdermal (topical), and transmucosal administration. Subjects may be administered by infusion or injection, by a single bolus or by repeated doses. Infusion or injection into organs and tissues (e.g., liver, spleen, heart, etc.). Specific examples of targeted delivery is injection or infusion into the portal vein, spleen or muscle.

For administration by inhalation, an aerosol spray from a pressured container or dispenser which contains a suitable propellant, e.g., a gas such as carbon dioxide, or a nebulizer can be employed. Transmucosal administration can be accomplished through the use of nasal sprays or inhalers.

The methods of the invention, including treating a bleeding or clotting disorder of a subject, likely results in an improvement in the subjects' condition, a reduction of symptoms or decreasing the subject's risk for developing symptoms of a bleeding or clotting disorder. Improvements therefore include one or more of decreased clotting time, reduced frequency of bleeding episodes or a reduced severity of bleeding episodes. An improvement may also be reducing the frequency or amount of a drug used for treating the subject for a bleeding or clotting disorder. For example, hemophilia B patients are treated by infusion with recombinant factor IX to decrease bleeding episodes. An improvement therefore would include reducing the dosage frequency or amount of recombinant factor IX that the subject was administered prior to treatment with a composition of the invention. An improvement may be relatively short in duration, e.g., several hours, days or weeks, or extend over a longer period of time, e.g., months or years. The improvement need not be a complete ablation of any or all symptoms of the disorder. For example, reducing severe hemophilia to a moderate or mild hemophilia is a satisfactory clinical endpoint. Thus, a satisfactory clinical endpoint is achieved when one or more of the aforementioned improvements in the subjects condition occurs, over a short or long duration.

Amounts of modified blood clotting factor sufficient to reduce or ameliorate one or more symptoms of the condition, e.g., for a bleeding or clotting disorder, decreased clotting time or reduced frequency or severity of bleeding, are generally significantly less than normal physiological levels of clotting factor. For example, achieving greater than 1% of normal plasma amounts of Factor IX can reduce clotting time and decrease the frequency of bleeding episodes in hemophilia B subjects (Kay et al., Nat. Genet. 24:257 (2000)). Thus, increasing the amount of Factor IX to greater than 1% of the normal level can reduce the frequency of injection of purified Factor IX. By analogy, an amount of modified blood clotting factor, such as Factor VIIa, sufficient to improve the subjects condition is therefore generally a small fraction of the levels present in a physiologically normal subject.

For Factor VII, plasma levels of recombinant Factor VIIa that achieve therapy are approximately 2-4 μg/ml. However, by continuously producing Factor VIIa in a subject using a method of the invention it is likely that less Factor VIIa will be needed to reduce symptoms associated with a bleeding or clotting disorder, or for prophylaxis. Accordingly, a therapeutic effect can be achieved with amounts of modified blood clotting factor significantly less than the amount used in recombinant Factor VIIa therapy.

Toxicity and therapeutic efficacy of a modified blood clotting factor can be determined using standard pharmaceutical procedures in cell cultures or test animals. For example, hemophilic mice are a well characterized model that mimic human hemophilia and is one particular example of a useful animal model for determining therapeutic dosage ranges. Hemophilia A knockout mice, one with a deletion of Factor VIII exon 16 and another with a deletion of exon 17, have less than 1% of normal circulating levels of Factor VIII (Bi and Lawler, Nature Genetics 10:119 (1995)). Hemophilia B knockout mice are also available, one type has a deletion of Factor IX through the third exon and appears to produce no detectable Factor IX (Sarkar et al., Hum. Gene Ther. 11:881 (2000)).

Hemophilac dogs are a second animal model useful for determining therapeutic dosage ranges. Dose calculations based on the hemophilac dog are a good approximation of a sufficient amount of modified blood clotting factor for humans. Doses that provide therapy in dogs have been used to begin clinical gene therapy trials in humans; dogs do appear to overestimate the amount required for humans. A dosage range of modified blood clotting factor about equal to the amount produced in the hemophilac dog which provides a therapeutic benefit to the dog is therefore an accurate measure of amounts expected to be sufficient for humans. At least three types of hemophilia B dogs are known in the art and are described, for example, in Evans et al., Proc. Natl. Acad. Sci. USA 86:10095 (1989); Mauser et al., Blood 88:3451 (1996); and Gu et al., Thromb. Haemost. 82:1270 (1999).

The dosage may vary within a broad range because therapeutic effects can be obtained with amounts that are significantly less than physiologically normal. While modified blood clotting factors that exhibit toxic side effects may be used, care should be taken to minimize potential side effects. Amounts of modified blood clotting factor in plasma can be determined directly by immunoaffinity detection or high performance liquid chromatography, or indirectly through an activity assay (e.g., prothrombin time) or by an improvement in the subjects' condition.

As discussed, an effective amount of modified Factor VIIa in the circulation ranges from about 14 μg/ml or higher, but may be less using a method of the invention. An effective amount of modified Factor Xa in the circulation ranges from about 10-100 ng/ml or higher, but may be less using a method of the invention. An effective amount of modified protein C in the circulation ranges from about 1-10% of normal levels or higher, but may be less using a method of the invention.

The delivery vehicle containing the modified blood clotting factor (e.g. vector, colloidal dispersion system or the like) can be administered to a subject as a single treatment or as a series of treatments, e.g., one time per week for between about 1 to 10 weeks. The skilled artisan will appreciate that certain factors may influence the dosage and timing required to provide an amount sufficient for therapeutic benefit. Such factors include but are not limited to the severity of the disorder, previous or simultaneous treatments, presence of inhibitors, the general health and/or age of the subject, and other diseases present.

The invention compositions and methods can be supplemented with other compositions and used in conjunction with other treatment/therapeutic protocols. For example, invention modified blood clotting factors can be combined with one or more drugs to treat a bleeding or clotting disorder.

The invention methods can be combined with other therapeutic protocols for treating a bleeding or clotting disorder. The invention methods can be performed prior to contemporaneously with or following treatment with another therapeutic protocol. Such drugs and therapeutic protocols are known in the art and include drugs and therapeutic protocols for treating hemophilia A, hemophilia B, Glanzmann's thrombasthenia and Bemard-Soulier's thrombasthenia.

Particular non-limiting examples of drugs useful in combination with invention compositions and methods include recombinant blood clotting factors (e.g., rFVIIa, rFVIII and rFIX) or blood fractions containing one or more supplementary clotting factors.

Patients that are likely to be treated with the invention compositions and by the invention methods may also have concomitant disorders requiring treatment with agents such as, but not limited to, antiviral drugs (e.g., for HIV) or interferon γ (for hepatitis). Patients with inhibitors may also be treated with immunosuppressive agents as pharmacological immunosuppression to induce immune tolerance in patients with high titer inhibitors has had success (Green, Blood 37:381 (1971) and Nilsson et al., Prog. Clin. Biol. Res. 324:69 (1990)). Transient immunomodulation prior to, contemporaneously with or following administering a modified blood clotting factor may provide a more effective treatment.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All publications, patents and other references cited herein are incorporated by reference in their entirety. In case of conflict, the present specification, including definitions, will control.

As used herein, the singular forms “a”, “and,” and “the” include plural referents unless the context clearly indicates otherwise. Thus, for example, reference to “a transformed cell” includes a plurality of such cells and reference to “a modified blood clotting factor” can include reference to one or more such factors, and so forth.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example describes the generation of modified FVIIa constructs.

A PCR-based mutagenesis protocol was used to generate 3 FVIIa constructs, as shown in FIG. 1. The amino acid inserts are: Arg-Lys-Arg (RKR), Arg-Lys-Arg-Arg-Lys-Arg (RKRRKR or 2xRKR for short) and Pro-Arg-Pro-Ser-Arg-Lys-Arg-Arg (PRPSRKRR, or INS for short).

In brief, human factor FVII cDNA (Hagen et al., Proc. Natl. Acad. Sci. USA 83:2412 (1986)) and cloned in the pCDNA3 vector as a BamH I fragment. Insertion of the amino acid cleavage recognition sequences at position Arg152-Ile153 was performed using the PCR mutagenesis kit (Stratagene) or by strand-overlap extension using appropriate primers. The sequence of each construct was verified by DNA sequencing.

### Example 2

This example describes expression of the modified FVIIa in cells. This example also describes data indicating that the modified FVII is cleaved at the inserted cleavage/recognition site to produce FVIIa and secreted by cells; and that the secreted protein can promote clotting. This example also describes data indicating that the modified FVII is autoactivate

The three vectors and wild type FVII were stably transfected into human embryonic kidney (HEK 293) cells. The highest-expressing single-cell clones (as assayed by a specific ELISA for human FVII, Enzyme Research Laboratories) were used for the production of recombinant FVIIa protein.

Serum-free conditioned medium from the FVII, FVII-RKR, FVII-2xRKR and FVII-INS clones grown in tissue culture, in the presence of vitamin K, were collected, concentrated and quantified by ELISA. No protease inhibitors were used in the tissue culture medium. 200 ng was electrophoresed and western blots analyzed cleavage of the protein using a sheep anti-human FVII antibody. As shown in FIG. 2A, the FVIIa protein secreted by HEK 293 cells is primarily in the active two-chain form (FVII-RKR, FVII-2xRKR and FVII-INS), although there was some single-chain material. These results indicate cleavage at the inserted sequence. In contrast, wild type FVII is secreted in a single-chain form indicating that secretion into the medium did not result in activation of the FVII molecule.

FVII, FVII-RKR, 2xRKR and FVII-INS proteins from the medium were also purified using Q-sepharose and an immunoaffinity column with a monoclonal anti-human FVII antibody in the presence of a protease inhibitor (to eliminate autoactivation). Four μg of purified protein was electrophoresed under reducing conditions and stained with coomassie blue. As shown in FIG. 2B, the purified proteins were in either a single-chain form (FVII) or primarily in double-chain form (FVIIa). N-terminal sequencing of the cleaved FVIIa indicated that intracellular cleavage had occurred at position Arg152Ile153.

Taken together, these results indicate that the secreted FVIIa proteins are cleaved at the inserted site intracellularly, and not in the medium into which they were secreted.

To demonstrate that FVII-RKR and FVII-2xRKR secreted by transfected HEK 293 cells is functional the FVII-RKR and FVII-2xRKR secreted in the tissue culture medium was assayed using a prothrombin time clotting assay; commercially available FVIIa (NovoSeven FVIIa) was used as a standard. Both FVII-RKR and FVII-2xRKR showed nearly identical clotting activity to NovoSeven FVIIa. Control medium (conditioned medium from untransfected cells) showed no clotting activity and wild-type FVII showed approximately 10% activity compared to NovoSeven FVIIa. Thus, both recombinant FVII-RKR and FVII-2xRKR shorten the prothrombin time in a one-stage clotting assay.

These results indicate that modified factor VII is effectively processed to activated factor VII (FVIIa), secreted into the media, and decreases prothrombin clotting time comparable to commercially available FVIIa.

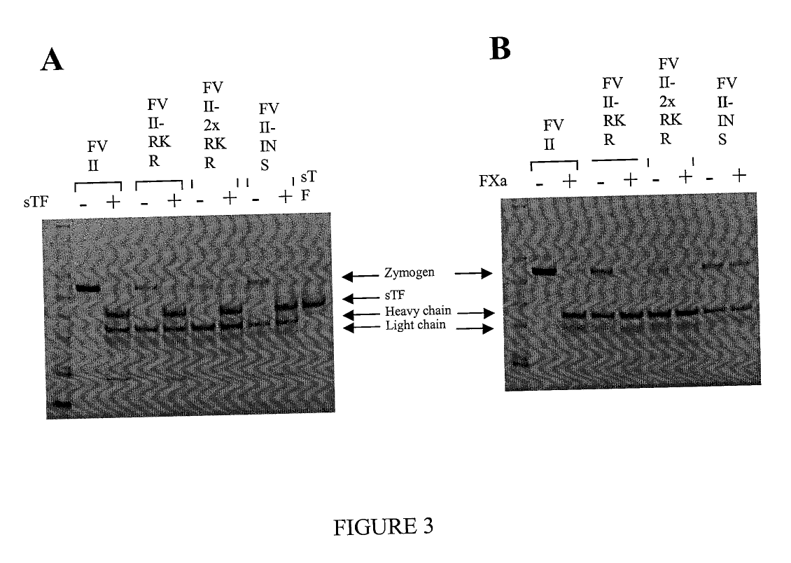

To address whether the single-chain material observed in the modified FVIIa preparations is a suitable substrate for cleavage, FVII autoactivation and FXa activation assays were performed. In brief, 2 μM of FVII-RKR, FVII-2xRKR and FVII-INS were incubated in the presence of soluble tissue factor, phospholipids and calcium for 2 h at 37° C. The products were electrophoresed in reducing conditions. In this reaction, single-chain protein (zymogen) in the FVII or the FVIIa preparations was cleaved to yield a two-chain material (FVIIa). The results in FIG. 3A demonstrate that all modified FVIIa contain single-chain material that can be cleaved.

For the FXa studies, the reaction was stopped by adding 21M tick anticoagulant protein (TAP) that specifically inhibits FXa activity. The products were electrophoresed as before. The results in FIG. 3B indicate that the single-chain zymogen protein in all but FVII-INS was nearly completely converted to two-chain material. Addition of 1 μM FXa (i.e 50 fold higher than for the other FVIIa proteins) resulted in complete cleavage of zymogen FVII-INS.

### Example 3

This example describes expression of modified FVII in a human hepatoma cell line.

Because liver is a particular target tissue for expression of modified blood clotting factors, expression of FVII was analyzed in HepG2 cells, a cell line previously reported to secrete FVII (Fair and Marlar, Blood 67:64 (1986)). To determine the amount of FVII secreted by this cell line and to confirm the published results HepG2 cells were cultured for 8 days continuously and part of the culture medium collected on day 2, 5 and 8. FVII was not reproducibly detected on days 2 and 5 but on day 8, approximately 25 ng/ml was detected in the medium, as reported by other investigators (Fair and Marlar, Blood 67:64 (1986)).

To demonstrate the ability of HepG2 cells transduced by recombinant AAV to secrete FVIIa, recombinant AAV vector with FVII-2xRKR under the control of the hAAT-ApoE promoter/enhancer was constructed. An rAAV vector backbone with a liver-specific enhancer/promoter (human α₁-antitrypsin and the apolipoprotein E enhancer, hAAT-ApoE) was used to drive expression of the modified FVIIa transgene. The HAAT-ApoE promoter has been shown to confer high-level expression in liver (Okuyama, et al., Hum. Gene Ther. 7:637 (1996)) and functions well in the context of as AAV vector (Herzog, et al, American Society of Hematology Meeting, San Francisco, Calif., U.S.A (2000)).

rAAV vector stocks were prepared using the triple transfection protocol (Matsushita, et al., Gene Ther. 5:938 (1998)). Target cells are seeded at a density that gave approximately a 30-50% confluency after 1 day, transduced with rAAV for 24 h, medium changed and FVIIa antigen measured by ELISA. Medium was assayed for modified FVIIa at day 2 following virus infection, changed and assayed again at day 5. A difference in FVIIa expression was detected at day 5 for multiplicity of infection (MOI) of 5*10³ and 50*10³; FVIIa expressed was 60 and 120 ng/ml/48 h, respectively.

These results demonstrate that a human hepatoma cell line can secret the cleaved/active form of modified FVII (FVIIa). These data also indicate that higher levels of FVIIa expression can be attained using higher MOIs.

### Example 4

This example describes data indicating that FVIIa can be measured in mouse plasma by ELISA. This example also describes data indicating that human FVIIa injected into a hemophilic mouse model can be monitored.

For the in vivo studies, the specificity and accuracy of the human FVII ELISA kit was evaluated. Normal mouse plasma was spiked with increasing amounts of NovoSeven FVIIa and was assayed by ELISA (Table 1, column 1). As shown in Table 1, column 2, FVIIa can be specifically detected in normal mouse plasma with relatively high accuracy.

TABLE 1

| | Observed human FVIIa |
|---|---|
| Sample | concentration (ng/ml) |
| Mouse plasma | Not detected |
| +100 ng/ml FVIIa | 90 |
| +500 ng/ml FVIIa | 560 |
| +1000 ng/ml FVIIa | 890 |
| +2500 ng/ml FVIIa | 2500 |
| +5000 ng/ml FVIIa | 5100 |

In order to demonstrate the efficacy of FVIIa treatment in an animal model for hemophilia, hemophilia A or B mice were injected with 90 μg/Kg (clinically effective dose) without inhibitors via the tail vein. Effectiveness of treatment was monitored by a one-stage clotting assay (prothrombin time, PT). Shortening of the PT by 5-7 seconds was observed in every case immediately after injection and subsequent return to baseline occurred within hours. These data demonstrate the ability to monitor human FVIIa treatment in a hemophilic mouse model, as well as the effectiveness of FVIIa treatment.

### Example 5

This example describes animal studies indicating that a viral expression vector can confer expression of modified FVIIa in vivo.

The recombinant AAV vector with the ApoE/hAAT driving expression of modified FVIIa transgene, described in Example 3, was used for mouse injections. In brief, normal male C57BL/6 or C57BL/6 Rag-i (immunodeficient) mice were injected with AAV-hAAT-ApoE-modified FVIIa at a dose of 1×10¹¹ vector genomes/animal into the portal vein using a Hamilton syringe (Nakai et al., Blood 91:4600 (1998)). Mice (a) and (b) were injected via the portal vein and mice (c), (d), (e) and (f) were injected into the spleen. Following injection, the peritoneal cavity was closed with 4-0 silk and the skin closed with 4-0 Vicryl.

Blood was collected at various time points after injection and modified FVIIa levels measured by ELISA (in ng/ml plasma). The results in Table 2 indicate that the modified FVIIa protein was continuously expressed in the mouse circulation.

TABLE 2

| AAV2 dose (×10¹¹) | Mouse strain | FVIIa (2 weeks) | FVIIa (4 weeks) | FVIIa (6 weeks) | FVIIa (8 weeks) |
|---|---|---|---|---|---|
| (a) 1 | C57BL/6 | Approx. 50 | Approx. 80 | Approx. 50 | Approx. 100 |
| (b) 0.7 | C57BL/6 | Approx. 50 | Approx. 50 | Approx. 40 | Approx. 100 |
| (c) 1.5 | C57BL/6 | 100 | ND | ND | ND |
| (d) 2 | C57BL/6 | 40 | ND | ND | ND |
| (e) 2 | Rag-1 | Approx. 85 | ND | ND | ND |
| (f) 1.5 | Rag-1 | Approx. 75 | ND | ND | ND |

ND: not determined

The animals were also examined for the presence of inhibitory antibodies (neutralizing and non-neutralizing) against the modified FVIIa. Mice (a) and (b) were assayed for neutralizing antibody against human FVIIa-2xRKR at week 2 and 4 and were found negative using a prothrombin-time based assay. Additionally, no non-neutralizing antibody at the same time points were detected using an ELISA-based assay.

These studies confirm that gene transfer of modified FVIIa using the AAV-HAAT-ApoE-FVIIa expression vector offers a treatment for hemophilia patients and does not appear to induce production of inhibitory antibodies against FVIIa.

What is claimed is:

1. A composition comprising a recombinant polynucleotide that encodes a modified blood clotting factor, wherein the modification comprises a proteolytic cleavage site not normally present in the factor, and wherein the factor is cleaved at the cleavage site when expressed in an animal cell.

2. The composition of claim 1, wherein the blood clotting factor is a functional variant or a functional subsequence of a naturally occurring blood clotting factor.

3. The composition of claim 1, wherein the blood clotting factor is a vitamin K-dependent procoagulent or anticoagulent protein.

4. The composition of claim 3, wherein the vitamin K-dependent procoagulent protein comprises Factor VII, Factor IX or Factor X.

5. The composition of claim 3, wherein the vitamin K-dependent anticoagulent protein comprises protein C.

6. The composition of claim 1, wherein the proteolytic cleavage site is a mammalian amino acid sequence.

7. The composition of claim 1, wherein the proteolytic cleavage site comprises a PACE/furin amino acid sequence, or functional variant thereof.

8. The composition of claim 1, wherein the proteolytic cleavage site comprises a plurality of basic amino acid sequences.

9. The composition of claim 1, wherein the proteolytic cleavage site comprises Arg-Lys-Arg, Arg-Lys-Arg-Arg-Lys-Arg (SEQ ID NO:1) or PRPSRKRR (SEQ ID NO:2) sequence.

10. The composition of claim 1, wherein the proteolytic cleavage site comprises a viral amino acid sequence cleavage site.

11. The composition of claim 10, wherein the viral cleavage site comprises a retroviral protein amino acid sequence.

12. The composition of claim 11, wherein the retroviral protein cleavage site is an envelope polypeptide cleavage site.

13. The composition of claim 4, wherein the proteolytic cleavage site is introduced between amino acids 152 and 153 of Factor VII.

14. The composition of claim 4, wherein the proteolytic cleavage site is introduced between arginine 152 and isoleucine 153 of Factor VII.

15. The composition of claim 1, wherein the animal cell is mammalian.

16. The composition of claim 15, wherein the mammalian cell is human.

17. The composition of claim 2, wherein the functional variant has one or more conservative amino acid substitutions of wild type blood clotting factor.

18. The composition of claim 2, wherein the functional variant comprises a Factor VII having increased activity relative to wild type Factor VII.

19. The composition of claim 2, wherein the functional variant comprises a Factor VII having increased stability in viva relative to wild type Factor VII.

20. The composition of claim 2, wherein the functional variant comprises a Factor VII having decreased immunogenicity relative to wild type Factor VII.

21. The composition of claim 1, wherein the Factor is mammalian.

22. The composition of claim 21, wherein the Factor is primate, canine, feline, porcine, equine or bovine.

23. The composition of claim 22, wherein the primate is human.

24. The composition of claim 1, wherein the recombinant polynucleotide encoding the modified blood clotting factor is operatively linked to a regulatable or tissue specific expression control element.

25. The composition of claim 24, wherein the regulatable or tissue specific expression control element comprises a promoter.

26. The composition of claim 24, wherein the promoter comprises a skeletal muscle actin promoter or a muscle creatine kinase promoter.

27. The composition of claim 24, wherein the tissue-specific expression control element confers expression of the modified blood clotting factor in muscle, liver, kidney or blood vessel endothelium.

28. The composition of claim 24, wherein the regulatable expression control element comprises elongation factor 1α promoter.

29. The composition of claim 1, further comprising a vector.

30. The composition of claim 29, wherein the vector comprises a vector suitable for introduction into a cell in vivo.

31. The composition of claim 30, wherein the vector comprises an adeno-associated virus (AAV), adenovirus, retrovirus, parvovirus, papilloma virus, reovirus, rotavirus or a herpes virus.

32. The composition of claim 30, wherein the vector comprises a plasmid vector.

33. A polypeptide encoded by the recombinant polynucleotide of claim 1.

34. A kit comprising a composition of claim 1 or a polypeptide of claim 33.

35. A kit comprising a composition of claim 1 further including instructions for expressing the modified blood clotting factor in vitro, ex vivo or in vivo.

36. The composition of claims 1 or 33, further comprising a cell.

37. The composition of claim 36, wherein the cell is a muscle, liver, kidney or blood vessel cell.

38. The composition of claim 36, wherein the cell is present in a subject.

39. The composition of claim 38, wherein the subject is a non-human transgenic animal.

40. The composition of claim 38, wherein the subject is human.

41. The composition of claims 1, further comprising a pharmaceutically acceptable carrier.

42. A method for treating a bleeding or clotting disorder of a subject having or at risk of having a bleeding or clotting disorder comprising administering to the subject an amount of the composition of claim 1 sufficient to ameliorate one or more symptoms of the disorder.

43. The method of claim 42, wherein the disorder is amenable to treatment with Factor VII, Factor VIII or Factor IX.

44. The method of claim 42, wherein the disorder is caused by insufficient activity or expression of a vitamin-K dependent procoagulent.

45. The method of claim 42, wherein the disorder is caused by insufficient platelet aggregation.

46. The method of claim 42, wherein the disorder comprises hemophilia or Factor VII deficiency.

47. The method of claim 46, wherein the hemophilia comprises hemophilia A or hemophilia B.

48. The method of claim 42, wherein the disorder comprises Glanzmann's thrombasthenia.

49. The method of claim 42, wherein the disorder comprises Bemard-Soulier's thrombasthenia.

50. The method of claim 42, wherein the subject produces inhibitory antibodies that bind to a clotting factor.

51. The method of claim 50, wherein the inhibitory antibodies bind Factor VIII or Factor IX.

52. The method of claim 42, wherein the subject is a mammal.

53. The method of claim 42, wherein the mammal is human.

54. The method of claim 42, wherein the composition is administered by injection or infusion.

55. The method of claim 42, wherein the composition is administered into the portal vein or spleen.

56. A method of decreasing clotting time in a subject in need of decreased clotting time comprising administering to the subject an amount of the composition of claim 1 sufficient to decrease clotting time in the subject.

57. The method of claim 56, wherein the modified blood clotting factor comprises Factor VII, Factor VIII or Factor IX.

58. The method of claim 56, wherein the subject is a mammal.

59. The method of claim 58, wherein the mammal is human.

60. A method of reducing the frequency or severity of bleeding in a subject in need of reduced frequency or severity of bleeding comprising administering to the subject an amount of the composition of claim 1 sufficient to reduce the incidence or severity of a bleeding in the subject.

61. The method of claim 60, wherein the composition comprises Factor VII, Factor VIII or Factor IX.

62. The method of claim 60, wherein the subject is a mammal.

63. The method of claim 62, wherein the mammal is a human.
